(19) United States
(12) Patent Application Publication
(10) Pub. No.: US 2025/0313783 A1
(43) Pub. Date: Oct. 9, 2025

(54) INHIBITION OF SACCHAROMYCES BY PICHIA PLUYVERI

(71) Applicant: CHR. HANSEN A/S, Hoersholm (DK)

(72) Inventors: Emilie Urban SLOTH, Hoersholm (DK); Simon CARLSEN, Hoersholm (DK); Camilla Maciel Rabelo PEREIRA, Hoersholm (DK); Jannie SCHNAB, Hoersholm (DK); Jesper Kjeldgaard ANDERSEN, Hoersholm (DK)

(73) Assignee: CHR. HANSEN A/S, Hoersholm (DK)

(21) Appl. No.: 18/872,399

(22) PCT Filed: Jun. 28, 2023
(86) PCT No.: PCT/EP2023/067691

§ 371 Date: Dec. 6, 2024

(30) Foreign Application Priority Data
Jun. 30, 2022 (EP) 22182260.4
Aug. 9, 2022 (EP) 22189520.4

(51) Int. Cl. C12C 12/00; C12C 12/04; C12N 1/16; C12R 1/84

(52) U.S. Cl. CPC C12C 12/006; C12C 12/04; C12N 1/165; C12R 2001/84

(57) ABSTRACT

The patent is related to use of one or more Pichia kluyveri strains for inhibiting or delaying growth of Saccharomyces spp. in beverages, preferably beverage with reduced alcohol content. Provided herein are also Pichia kluyveri deposited as DSM 34278 and Pichia kluyveri deposited as DSM 34279 and mutants thereof, as well as composition comprising said Pichia kluyveri.

### FIELD OF THE INVENTION

The present invention lies in the field of microbiology and relates to methods for inhibiting or delaying growth of Saccharomyces spp. in the production of beverages particularly those low in alcohol, in particular fermented beverages like beer with lower alcohol content.

### BACKGROUND OF THE INVENTION

Due to the increasing demand for healthier food and beverages, the reduction of ethanol in alcoholic beverages, especially beer and wine, is of considerable commercial interest. Specifically, alcohol-free beer as a malt-based, calorie-reduced refreshment with various nutrient-physiologically positive properties is becoming more popular.

Compared to standard beers which normally contain more than 4% (vol/vol) alcohol, low alcohol beer or non-alcoholic beer (also called light beer, no-alcohol, nonalcoholic beer, small beer, small ale, near-beer) are beers with lower alcohol content which aim to reproduce the full flavor of standard beers.

Various processes are known and already established for the production of beer with a reduced alcohol content, low alcohol beer and non-alcoholic beer. In general, these processes can be classified into two groups. On one hand, there are physical processes in which ethanol is removed as selectively as possible from regular beer or another alcoholic beverage matrix such as wine or cider, mainly by thermal or membrane processes. On the other hand, there are biological methods, which are based on restricted ethanol formation, such as use of a modified mashing process, use of a limited fermentation process such as stopped fermentation, or use of special yeasts.

The conversion of wort into an alcoholic beer is the result of a fermentation process by Saccharomyces spp. yeast resulting in the production of alcohol and fermentation-derived flavor compounds, such as esters and higher alcohols.

However, the micro-environment of breweries and brewing tanks are normally highly dominated by Saccharomyces spp., including S. cerevisiae and S. pastorianus which are used in the breweries, as well as other Saccharomyces which are contaminants such as Saccharomyces diastaticus. It is difficult to completely eliminate these from brewing tanks by cleaning and disinfection. This can pose a problem for the production of low alcohol beer and non-alcoholic beer. When brewing this type of beer, any contamination with Saccharomyces spp. may result in ethanol production and thereby exceeding the targeted alcohol concentration, as well as potential off-flavors or other deteriorations in quality. Spoilage of Saccharomyces in beer is for example described in Suiker et al. “Spoilage yeasts in beer and beer products.” Current Opinion in Food Science 44 (2022): 100815. The lower the allowed residual alcohol in the beer, the more critical is the inhibition of Saccharomyces spp. in the process. Thus, when using biological methods for the production of alcohol reduced beer, it is imperative to suppress or fully inhibit the growth of Saccharomyces spp. that may be present in the fermentation tanks.

Thus, there is a need for an improved method for inhibiting the growth of Saccharomyces spp. when producing beverages, in particular beverages susceptible to Saccharomyces contamination. Specifically, it would be desirable to provide an efficient inhibition of Saccharomyces spp. during the production of a beverage while preferably providing for a beverage having good organoleptic or sensory properties, such as a good aroma or flavor profile.

### SUMMARY OF THE INVENTION

This problem is now solved by the use of one or more Pichia kluyveri strains for inhibiting or delaying growth of Saccharomyces spp. in a beverage.

Though the use of Pichia kluyveri to make low-alcohol beer is known from e.g. WO 2014/135673 which discloses the use of Pichia kluyveri strains PK-KR1 and PK-KR2, no inhibition of Saccharomyces spp. was disclosed.

The one or more Pichia kluyveri strains in the present invention is characterized by the ability to inhibit growth of S. cerevisiae or S. pastorianus by at least 40%, such as at least 50%, at least 60%, at least 70%, or at least 80%, when the S. cerevisiae or S. pastorianus is inoculated simultaneously with the one or more Pichia kluyveri strains, compared to an otherwise identical sample which does not comprise the one or more Pichia kluyveri strains.

The inventors of the present invention have surprisingly found new methods to manage Saccharomyces spp. contamination by using Pichia kluyveri. Thus, the invention contributes to provide an effective solution to manage Saccharomyces spp. growth by biological means and may avoid the use of chemical disinfectants.

The use and methods of the present invention are particularly suitable for fermenting low- or non-alcoholic beverages. Saccharomyces spp. are commonly present in the microenvironment of breweries. Since these yeasts are difficult to completely eliminate from brewing tanks by cleaning and disinfection, it is highly advantageous to use a Pichia kluyveri strain to efficiently inhibit growth of Saccharomyces spp. contamination present in the system. With the use and methods of the present invention it is possible to inhibit growth of Saccharomyces present in the fermentation tank and preferably that the inhibition is not present at the next batch of production in the same the fermentation tank for regular beer brewing. This has the advantage that the breweries do not need to have dedicated tanks for non-alcoholic beer production.

In particular, the present inventors have identified two novel Pichia kluyveri strains (DSM 34278 and DSM 34279) which are particularly suitable for the use and methods of the present invention, as they are able to efficiently inhibit Saccharomyces spp. Furthermore, when used in fermenting non-alcoholic beverage, these strains additionally provide a very good flavor profile.

To combat the problem of microbial contamination, the present invention provides in a first aspect a use of one or more Pichia kluyveri strains for inhibiting or delaying growth of Saccharomyces spp. in a beverage, preferably fermented beverages. The one or more Pichia kluyveri strains can be characterized in that it is able to inhibit growth of S. cerevisiae or S. pastorianus by at least 40% when inoculated simultaneously with the one or more Pichia kluyveri strains, compared to an otherwise identical sample which does not comprise the one or more Pichia kluyveri strains. Preferably, the Pichia kluyveri is added to the beverage at a concentration of at least 10³ CFU/ml, such as at least 10⁴ CFU/ml, such as at least 10⁵ CFU/ml, such as 10³ to 10⁹ CFU/ml, preferably 10⁴ to 10⁸ CFU/ml, most preferably 10⁷ CFU/ml.

In a second aspect, the present invention provides a method of inhibiting or delaying growth of Saccharomyces spp. in a beverage, preferably fermented beverages, more preferably a beverage with lower alcohol content, such as with alcohol content of less than 4.2 vol.-%, comprising the step of adding an effective amount of one or more Pichia kluyveri strains. The one or more Pichia kluyveri strains can be characterized in that it is able to inhibit growth of S. cerevisiae or S. pastorianus by at least 40%, such as at least 50%, at least 60%, at least 70%, or at least 80%, when inoculated simultaneously with the one or more Pichia kluyveri strains, compared to an otherwise identical sample which does not comprise the one or more Pichia kluyveri strains. Preferably, the Pichia kluyveri is added to the beverage at a concentration of at least 10³ CFU/ml, such as at least 10⁴ CFU/ml, such as at least 10⁵ CFU/ml, such as 10³ to 10⁹ CFU/ml, preferably 10⁴ to 10⁸ CFU/ml, most preferably 10⁷ CFU/ml.

In a third aspect, the present invention provides a yeast of the species Pichia kluyveri deposited as DSM 34278 or a mutant Pichia kluyveri obtainable from the deposited yeast. The mutant may be characterized by an ability to inhibit growth of S. cerevisiae or S. pastorianus by at least 40%, such as at least 50%, at least 60%, at least 70%, or at least 80%, when the Saccharomyces is inoculated simultaneously with the Pichia kluyveri strain, compared to an otherwise identical sample which does not comprise the Pichia kluyveri strain, determined using an assay, for example Assay I.

In a fourth aspect, the present invention provides a yeast of the species Pichia kluyveri deposited as DSM 34279 or a mutant Pichia kluyveri obtainable from the deposited yeast. The mutant may be characterized by an ability to inhibit growth of S. cerevisiae or S. pastorianus by at least 40%, such as at least 50%, at least 60%, at least 70%, or at least 80%, when the Saccharomyces is inoculated simultaneously with the Pichia kluyveri strain, compared to an otherwise identical sample which does not comprise the Pichia kluyveri strain, determined using an assay, for example Assay I.

In further aspects, the present invention provides a composition or a beverage comprising the yeast of the third and/or fourth aspect of the present invention, in particular a beverage with lower alcohol content such as less than 4.2 vol.-%.

### DETAILED DISCLOSURE OF THE INVENTION

Unless defined otherwise, all technical and scientific terms used herein have the same meaning as commonly understood by persons skilled in the art. Although any methods and materials equivalent or similar to those described herein can be used in the practice of the present disclosure, typical methods and materials are described. The detailed information provided for the use of the present invention relates to the methods of the invention as well.

The present invention provides in a first aspect use of one or more Pichia kluyveri strains for inhibiting or delaying growth of Saccharomyces spp. in a beverage, wherein the Pichia kluyveri strain is characterized in that it is able to inhibit growth of S. cerevisiae or S. pastorianus by at least 40% when the Saccharomyces is inoculated simultaneously with the Pichia kluyveri strain, compared to an otherwise identical sample which does not comprise the Pichia kluyveri strain.

As used herein, the terms “to inhibit” and “inhibiting” in relation to Saccharomyces spp. mean that the growth, the number, or the concentration of Saccharomyces spp. is reduced. Inhibition can be observed by comparing the growth, number, or concentration of Saccharomyces in the product with the Pichia kluyveri strain to a control. The control can be the otherwise identical composition but without the Pichia kluyveri strain. Yeast growth can be measured with various methods known to a skilled person in the art. Methods of determining yeast growth inhibition or delay are known to a skilled person in the art. For example, yeast growth can be measured by quantifying the amount of yeast in a sample after a given time using real-time PCR (qPCR) assay and comparing the determined amount to a negative control.

The term “to delay” in general means the act of stopping, postponing, hindering, or causing something to occur more slowly than normal. As used herein, “delaying growth of Saccharomyces spp.” refers to the act of postponing the growth of Saccharomyces spp. This can be observed by comparing the time needed for the Saccharomyces spp. to grow to a given level in two otherwise identical beverages, one of which with the Pichia kluyveri strain and the other one without.

In some embodiments, “delaying growth” refers to delaying by 1 day, such as 2, 3, 4, 5, 6, 7, 8, 9, 10, 15, 20 days.

In a preferred embodiment, the growth of Saccharomyces spp. in the beverage is inhibited by at least 40%, preferably at least 50% or 75%, compared to an otherwise identical sample which does not comprise the one or more Pichia kluyveri strains. This is for example illustrated in Example 1 and 2. A beer Saccharomyces can be used as target microorganism.

The inhibition may be determined by Assay I:

### Assay I:

inoculating wort (8° P and 13 IBU) with/without a starting concentration of Pichia kluyveri of 10⁵ CFU/ml,
fermenting the wort at 18° C. at 120 rpm for 3 days, and
comparing the growth of the S. cerevisiae in the wort medium with/without Pichia kluyveri by qPCR and determining the percentage of inhibition.

The inventors of the present invention have surprisingly discovered that Saccharomyces spp. can be inhibited by an effective amount of Pichia kluyveri. Strains of Pichia kluyveri, which may be used in the present invention, include Pichia kluyveri DSM 34278, Pichia kluyveri DSM 34279, Pichia kluyveri DSM 28484, Pichia kluyveri CBS188, mutants thereof and mixtures thereof. The mutants are functionally equivalent mutants that have the same or improved ability to inhibit or delay growth of Saccharomyces spp. compared to their respective mother strain. The ability can be determined using an assay such as Assay I.

In the present context, the term “mutant” should be understood as a strain derived from a strain of the invention, for example by means of e.g. genetic engineering, radiation and/or chemical treatment. It is preferred that the mutant is a functionally equivalent mutant, e.g. a mutant that has substantially the same, or improved, properties in particular in relation to the effects on inhibiting growth of Saccharomyces spp, as the deposited strain. Respective mutants represent embodiments of the present invention. The term “mutant” in particular refers to a strain obtained by subjecting a strain of the invention to any conventionally used mutagenization treatment including treatment with a chemical mutagen such as ethane methane sulphonate (EMS) or N-methyl-N′-nitro-N-nitroguanidine (NTG), UV light or to a spontaneously occurring mutant. A mutant may have been subjected to several mutagenization treatments (a single treatment should be understood one mutagenization step followed by a screening/selection step), but it is presently preferred that no more than 20, or no more than 10, or no more than 5, treatments (or screening/selection steps) are carried out. In a presently preferred mutant, less than 5%, or less than 1% or even less than 0.1% of the nucleotides in the bacterial genome have been shifted with another nucleotide, or deleted, compared to the mother strain.

In a preferred embodiment, the Pichia kluyveri strain used herein is not PK-KR1 or PK-KR2.

In a particularly preferred embodiment, the Pichia kluyveri strain is selected from the group consisting of strains deposited as DSM 34278 and DSM 34279 or a mutant thereof.

A Pichia kluyveri strain useful in the present invention can be characterized by its ability to inhibit growth of S. cerevisiae or S. pastorianus by at least 40%, such as at least 50%, at least 60%, at least 70%, or at least 80%, when the Saccharomyces is inoculated simultaneously with the Pichia kluyveri strain, compared to a negative control (i.e. an otherwise identical sample which does not comprise the Pichia kluyveri strain).

In a preferred embodiment, the Pichia kluyveri strain is characterized in that it inhibits growth of S. cerevisiae or S. pastorianus by at least 40% when inoculated simultaneously with the Pichia kluyveri strain in wort, compared to an otherwise identical wort which does not comprise the strains.

In a preferred embodiment, the Pichia kluyveri strain is able to inhibit growth of 10³ CFU/ml Saccharomyces spp. by at least 40% when the Saccharomyces is inoculated simultaneously with 10⁵ CFU/ml Pichia kluyveri strain, compared to an otherwise identical sample which does not comprise the Pichia kluyveri strain.

In a preferred embodiment, the one or more Pichia kluyveri strains is able to inhibit growth of 10³ CFU/ml Saccharomyces spp. by at least 40% when the Saccharomyces is inoculated simultaneously with 10⁵ CFU/ml Pichia kluyveri strain in wort for three days, compared to an otherwise identical wort which does not comprise the Pichia kluyveri strain.

In a preferred embodiment, the Pichia kluyveri strain is added to the fermented beverage preferably at the start or during fermentation, at a concentration of at least 10³ CFU/ml, such as at least 10⁴ CFU/ml, such as at least 10⁵ CFU/ml, such as 10³ to 10⁹ CFU/ml, preferably 10⁴ to 10⁸ CFU/ml, most preferably 10⁷ CFU/ml.

In a preferred embodiment, the growth of one or more of Saccharomyces cerevisiae, Saccharomyces carlsbergensis, Saccharomyces eubayanus, Saccharomyces bayanus, Saccharomyces uvarum, Saccharomyces kudriavzevii and/or Saccharomyces pastorianus is inhibited.

### Beverage

The beverage in which the growth of Saccharomyces spp. is inhibited or delayed can be any beverage, such as wine, cider, tea, fruit beverage or beer, preferably a beverage obtainable by fermentation, and most preferably obtainable by fermentation of wort, including wort-based beverages such as beer. The beverage is preferably with lower alcohol content, such as with alcohol content of less than 4.2 vol.-% or less.

As used in the present description, the term “beer” refers, at least, to the types of beer obtained from brewed malt obtained from malted grain, as well as brewed malt obtained from unmalted grain, and brewed malt obtained from a mixture of malted and unmalted grains.

The term “wort” herein has the conventional meaning in the art and refers to the sugary liquid extracted from the mashing process of beer brewing.

The term “fermentation” herein refers to a metabolic process that includes chemical changes in the wort substrate through the action of the culture. This includes aerobic and anaerobic processes.

The beverage is a beverage which is low in alcohol, which may be a beverage with a reduced alcohol content, low-alcohol beverage, an alcohol-free beverage or a non-alcoholic beverage. Meaning of such terms may differ according to national regulations, however, as used herein, In the present context the term “beverage with a reduced alcohol content” in the present description refers to a beverage with an alcohol content of 1.2 to 4.2 vol.-%. The term “low-alcohol beverage” herein is defined as a liquid for drinking with an alcohol content of more than 0.5 vol.-% and no more than 1.2 vol.-%. The term “non-alcoholic beverage” herein is defined as a liquid for drinking with an alcohol content of no more than 0.5 vol.-%. The term “alcohol-free beverage” herein is defined as a liquid for drinking with an alcohol content of less than 0.05 vol.-%. In one embodiment the beverage is wine that is low in alcohol.

### Beer

In a preferred embodiment, the beverage is a beer with a reduced alcohol content, a low alcohol beer, a non-alcoholic beer, or alcohol-free beer. Reduced means that the alcohol content is lower compared to normal beer. In the present context the term “beer with a reduced alcohol content” in the present description refers to beer with an alcohol content of 1.2 to 4.2 vol.-%. The term “low alcohol beer” as used herein refers to beer with an alcohol content of 0.5 to 1.2 vol.-%. The term “non-alcoholic beer” in the present description refers to beer with an alcohol content of less than 0.5 vol.-%. Finally, the term “alcohol-free beer” herein refers to beer with an alcohol content of less than 0.05 vol.-%.

### Fruit Beverage

In another preferred embodiment, the beverage is a fruit beverage. In the present context the term “fruit beverage” refers to a beverage comprising fruit juice, fruit concentrate and/or fruit puree. The term “fruit beverage” covers “fruit juice”, “fruit drink” and “fruit nectar”. The “fruit beverage” may be either one containing pulp, or one from which the pulp has been removed by such an operation as centrifugation. The fruit beverages may further contain e.g. oat, soy, almond, whey and/or non-fermented milk, e.g. in the form of milk powder.

The fruit beverages suitable for the use in the present invention may e.g. be fruit juices, fruit juices from concentrate, fruit drinks, fruit smoothies, and fruit nectar optionally comprising fruit purees and/or water.

The term “fruit juice” refers to the liquid naturally contained in fruit prepared by mechanically squeezing or macerating fresh fruits without the presence of heat and solvents. The “fruit juice” may consist of juice from one type of fruit or a mixture of more than one type of fruit.

The term “fruit drink” in the present context refers to a beverage having a fruit juice content of between 0 to 29 vol.-%.

The term “fruit nectar” in the present context refers to a beverage having a fruit juice content of between 30 to 99 vol.-% fruit juice.

In the present context the term “fruit puree” refers to fruits prepared by grounding, pressing and/or straining into the consistency of a thick liquid or a soft paste without the presence of heat and solvents. “Puree” is made of 100% fruit as opposed to being made from just the juice of the fruit.

The total content of fruit juice and/or fruit puree in the fruit beverage is generally between about 20% to about 99.99% by weight, preferably between about 30% to 95% by weight, more preferably between about 40% to 90% by weight, still more preferably between about 50% to 80% by weight, and most preferably 60% to 70% by weight.

### Methods of the Present Invention

In a second aspect, the present invention provides a method of inhibiting or delaying growth of Saccharomyces spp. in a beverage with lower alcohol content, such as with alcohol content of less than 4.2 vol.-%, comprising the step of adding an effective amount of one or more Pichia kluyveri strains to the beverage, wherein the one or more Pichia kluyveri strains is characterized in that it is able to inhibit growth of S. cerevisiae or S. pastorianus by at least 40% when the Saccharomyces is inoculated simultaneously with the one or more Pichia kluyveri strains, compared to an otherwise identical sample which does not comprise the one or more Pichia kluyveri strains.

The Pichia kluyveri strain may be added in any form, including the form of a liquid, frozen or dried (such as freeze-dried or spray dried) compositions. The composition preferably comprises one or more of cryoprotectants, lyoprotectants, antioxidants and/or nutrients, more preferably cryoprotectants, lyoprotectants and/or antioxidants and most preferably cryoprotectants or lyoprotectants, or both. Use of protectants such as cryoprotectants and lyoprotectants are known to a skilled person in the art. Suitable cryoprotectants or lyoprotectants (including mono-, di-, tri- and polysaccharides), polyols, amino acids, complex substances and inorganic compounds. The composition may optionally comprise further substances including emulsifiers (such as sorbitan monostearate), fillers and/or flavorants.

In preferred embodiments, the composition comprises at least 10⁶ CFU/g, including at least 10⁷ CFU/g, at least 10⁸ CFU/g, at least 10⁹ CFU/g, at least 1010 CFU/g, at least 1011CFU/g Pichia kluyveri.

Method of preparing high density yeast composition is known in the art and for example described in WO2011/134952.

In a further aspect, the present invention provides a method of producing a beverage with lower alcohol content, such as with alcohol content of less than 4.2 vol.-%, comprising the steps of adding an effective amount of one or more Pichia kluyveri strains to inhibit or delay growth of Saccharomyces spp. in the beverage, wherein the one or more Pichia kluyveri strains is characterized in that it is able to inhibit growth of S. cerevisiae or S. pastorianus by at least 40% when the Saccharomyces is inoculated simultaneously with the one or more Pichia kluyveri strains, compared to an otherwise identical sample which does not comprise the one or more Pichia kluyveri strains.

When using a yeast strain in a method of producing a beverage, the skilled person is able to adjust various parameters such as pH, temperature, and amount of yeast to achieve the desired results, taking into consideration the examples provided in this invention as well as the properties of the beverage to be produced.

The methods disclosed herein are particularly useful to inhibit or delay yeast growth in fermentation process, such as the production of a beer with a reduced alcohol content, a low alcohol beer, a non-alcoholic beer or alcohol-free beer.

### Yeasts and Compositions of the Present Invention

In third aspect, the present invention provides a yeast of the species Pichia kluyveri deposited as DSM 34278 or a mutant Pichia kluyveri obtainable from the deposited yeast, wherein the mutant is characterized by an ability to inhibit growth of S. cerevisiae or S. pastorianus by at least 40%, such as at least 50%, at least 60%, at least 70%, or at least 80%, when the Saccharomyces is inoculated simultaneously with the Pichia kluyveri strain, compared to an otherwise identical sample which does not comprise the Pichia kluyveri strain.

In a fourth aspect, the present invention provides a yeast of the species Pichia kluyveri deposited as DSM 34279 or a mutant Pichia kluyveri obtainable from the deposited yeast, wherein the mutant is characterized by an ability to inhibit growth of S. cerevisiae or S. pastorianus by at least 40%, such as at least 50%, at least 60%, at least 70%, or at least 80%, when the Saccharomyces is inoculated simultaneously with the Pichia kluyveri strain, compared to an otherwise identical sample which does not comprise the Pichia kluyveri strain.

In a preferred embodiment, the inhibition is tested using a starting concentration of Pichia kluyveri of 10⁵ CFU/ml and a starting concentration of S cerevisiae or S. pastorianus of 10³ CFU/ml. The inhibition is preferably tested in wort medium as described in the Example.

In a preferred embodiment, the inhibition is tested in wort after three days using a starting concentration of Pichia kluyveri of 10⁵ CFU/ml and a starting concentration of S cerevisiae or S. pastorianus of 10³ CFU/ml.

In a fifth aspect, the present invention provides a composition comprising the yeast of the third and/or fourth aspect.

The composition of the present invention may additionally comprise cryoprotectants, lyoprotectants, antioxidants, nutrients, fillers, flavorants or mixtures thereof. The composition may be in any form, including liquid (such as slurry), frozen or dried form (such as freeze dried or spray dried). The composition preferably comprises one or more of cryoprotectants, lyoprotectants, antioxidants and/or nutrients, more preferably cryoprotectants, lyoprotectants and/or antioxidants and most preferably cryoprotectants or lyoprotectants, or both. Use of protectants such as cryoprotectants and lyoprotectants are known to a skilled person in the art. Suitable cryoprotectants or lyoprotectants include mono-, di-, tri- and polysaccharides (such as glucose, mannose, xylose, lactose, sucrose, trehalose, raffinose, starch and gum arabic (acacia) and the like), polyols (such as erythritol, glycerol, inositol, mannitol, sorbitol, threitol, xylitol and the like), amino acids (such as proline, glutamic acid), complex substances (such as skim milk, peptones, gelatin, yeast extract) and inorganic compounds (such as sodium tripolyphosphate). Suitable antioxidants include ascorbic acid, citric acid and salts thereof, gallates, cysteine, sorbitol, mannitol, maltose. Suitable nutrients include sugars, amino acids, fatty acids, minerals, trace elements, vitamins (such as vitamin B-family, vitamin C). The composition may optionally comprise further substances including emulsifiers (such as sorbitan monostearate), fillers (such as lactose) and/or flavorants.

In a preferred embodiment, the composition comprises at least one cryoprotectant or lyoprotectants. In another embodiment, the composition comprises sorbitan monostearate. In a preferred embodiment, the composition is liquid or freeze-dried.

The use of the terms “a” and “an” and “the” and similar referents in the context of describing the invention (especially in the context of the following claims) are to be constructed to cover both the singular and the plural, unless otherwise indicated herein or clearly contradicted by context. The terms “consisting,” “possessing,” “including,” and “containing” should be interpreted as non-limiting terms (i.e., meaning “including but not limited to”), unless otherwise indicated.

### FIGURES

FIG. 1 shows the total concentration of S. cerevisiae expressed as gene copies/mL found in the fermentations at different timepoints (Day 1, Day 3, and Day 6)

FIG. 2 shows the total concentration of S. pastorianus expressed as gene copies/mL found in the fermentations at different timepoints (Day 1, Day 3, and Day 6).

FIG. 3 shows the concentration of ethyl acetate in beer samples prepared with Pichia kluyveri DSM 34278, DSM 34279 and DSM28484 measured by GC-MS.

FIG. 4 shows the concentration of off-flavor compound dihydrogen sulphide in beer samples prepared with Pichia kluyveri DSM 34278, DSM 34279 and DSM28484 measured by GC-MS.

FIG. 5 shows the concentration of off-flavor compound methional in beer samples prepared with Pichia kluyveri DSM 34278, DSM 34279 and DSM28484 measured by GC-MS.

### EXAMPLES

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description and the following examples. Such modifications are also intended to fall within the scope of the appended claims. In the case of conflict, the present disclosure including definitions will prevail.

### Example 1: Inhibition of Saccharomyces cerevisiae

### 1.1 Preparation of Samples

Saccharomyces cerevisiae (Safale US-05, Fermentis) was cultivated in malt media (8° P, prepared from Barlex 7215, Harboes Bryggeri A/S, Denmark; pH=4.4) at 25° C. at 150 rpm for 48 hours prior to inoculation.

Afterwards, wort of 8°P, 13 IBU was prepared from hopped (Magnum 12.9% AA) malt extract (Barlex 7215) and pH adjusted to 4.4. It was divided into 200 mL volume per sample in bluecap bottles. The samples were inoculated with or without various Pichia kluyveri strains simultaneously with Saccharomyces cerevisiae (Safale US-05, Fermentis). For an overview of the inoculations see Table 1. Origin of the strains are shown in Table 2. The fermentations were incubated at 18° C. at 120 rpm for 10 days. Sample of 10 mL was collected on day 1, 3 and 6 after inoculation and subsequently frozen at −50° C.

TABLE 1
Overview of fermentations and inoculation regime
with P. kluyveri and S. cerevisiae

| | | 1 | | | | | |
|---|---|---|---|---|---|---|---|
| Microorganism | Strain | Control | 2 | 3 | 4 | 5 | 6 |
| Pichia kluyveri | DSM 28484 | | 1E+05 | | | | |
| Pichia kluyveri | DSM 34278 | | | 1E+05 | | | |
| Pichia kluyveri | DSM 34279 | | | | 1E+05 | | |
| Pichia kluyveri | PK-KR1 | | | | | 1E+05 | |
| Pichia kluyveri | CBS188 | | | | | | 1E+05 |
| Saccharomyces | Safale US-05 | 1E+03 | 1E+03 | 1E+03 | 1E+03 | 1E+03 | 1E+03 |
| cerevisiae | | | | | | | |

TABLE 2
Origin of the strains

| Microorganism | Strain | Origin |
|---|---|---|
| Pichia kluyveri | DSM 28484 | Grape |
| Pichia kluyveri | DSM 34278 | Cocoa |
| Pichia kluyveri | DSM 34279 | Cocoa |
| Pichia kluyveri | PK-KR1 | Grape juice |
| Pichia kluyveri type strain | CBS188 | Olives |

### 1.2 Analysis of Yeast Growth

1.2.1 DNA Extraction from Fermentation Samples

Yeast cells were first isolated from 12 mL of Day 1 fermentations and 5 mL of Day 3 and Day 6 fermentations by centrifugation at 5.000×g for 10 min. The supernatant was discarded, and the cells were resuspended and washed in 500 μl buffer (1.2 M Sorbitol; 100 mM TRIS; 100 mM CaCl₂), 4 H₂O). Fermentation pellets were again centrifuged for 10 min at 5.000×g and supernatant discarded. Subsequently, 200 μl of lysis buffer (1.2 M Sorbitol; 100 mM TRIS; 100 mM CaCl₂), 4 H₂O, 200 U of liquid Lyticase) was added to the pellet and mixed vigorously by pipetting. Finally, samples were loaded onto the QIAcube connect instruments (Qiagen, Hilden, Germany) and DNA purified with Qiagen DNeasy Blood & Tissue Kit (Qiagen, Hilden, Germany) as per the manufacturer's instructions at final elution volume of 125 μl.

1.2.2 Real-Time PCR (qPCR) Assays

Primer specificity was assessed using Primer-blast comparisons, in silico primer-target (CLC Genomics Workbench v.20 Qiagen), and real-time PCR experimental validations using pure DNA from respective strains in fermentations. Primers specific to Saccharomyces sensu stricto complex were used to target and quantify only Saccharomyces cerevisiae (Safale US-05, Fermentis) and S. pastorianus (SmartBev, Chr. Hansen) in the fermentation samples. Primers SI4F: 5′-ATTGCTGGCCTTTTCATTG-3′ (SEQ ID NO: 1), SI7R: 5′-CGCCTAGACGCTCTCTTCTTAT-3′ (SEQ ID NO: 2) (Chang et al. “Quantitative real time PCR assays for the enumeration of Saccharomyces cerevisiae and the Saccharomyces sensu stricto complex in human feces.” Journal of microbiological methods 71.3 (2007): 191-201) and S-probe: FAM-5′-GCCGCAGTTGGTAAAACCTA-3′-BHQ (SEQ ID NO: 3) were designed to anneal within the ITS1-5.8S-ITS2 rDNA region and amplify products 163 bp in length. Real-time PCR amplification and detection were performed in an QuantStudio™ 12K Flex Real-Time PCR System (Applied Biosystems, Foster City, Calif., USA). The probe-based reaction mix was composed of 6.25 μL 1×qPCR™ Master Mix Plus-Low ROX (Eurogentec, Seraing, Belgium), 0.3 μM nmol of each forward and reverse primer, 0.6 UM of probe, 2.5 μl of 10× and 100× dilute template DNA (to avoid inhibitor effects), and molecular grade H₂O (Invitrogen, Life Technologies, Paisley, UK) to a final volume of 12.5 μL. The amplification program consisted of one cycle of 50° C. for 2 min, one cycle of 95° C. for 10 min, then 40 cycles of 95° C. for 15s, and 60° C. for 1 min. Standard curves were constructed using the DNA from pure culture of S. pastorianus (SmartBev, Chr. Hansen), and generated every time a qPCR analysis was performed, in parallel with the amplification of fermentation samples. Serial dilution (10-fold) of the standards was performed to obtain standard solutions in the range of 10⁸-101 pure DNA starting from 5 ng/μL. All samples were run in triplicate and molecular grade H₂O replaced template in control reactions.

### 1.3 Results

In total, 18 samples belonging to the different fermentations and controls taken at days 1, 3 and 6 were analyzed. To quantify S. cerevisiae in the experiment conducted on the fermentations, qPCR primers SI4F/SI7R and S-probe were used to amplify appropriate product size located on the ITS rRNA gene. The specificity of the primers and probe was validated by qPCR using genomic DNA from all yeast listed in Table 1. The assay displayed excellent efficiency (95% amplification efficiency, R² 0.998) and specificity for S. cerevisiae, whereas the various Pichia kluyveri strains remain under the lowest reliable detection limit.

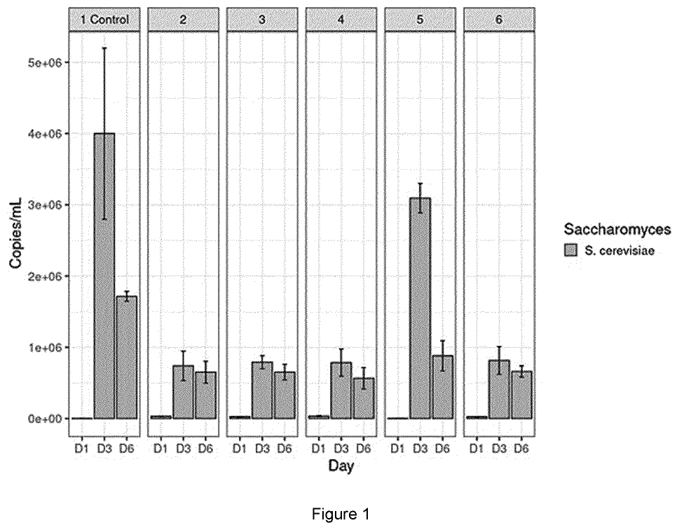

FIG. 1 shows the total concentration of S. cerevisiae expressed as gene copies/mL found in the fermentations after one day, three days and 6 days. A decrease of Saccharomyces growth between Day 3 and Day 6 was observed, compared to the control batch (without Pichia kluyveri). On the other hand, no growth inhibition of Saccharomyces occurred when the fermentation takes place with P. kluyveri PK-KR1.

These results show the effective use of various Pichia kluyveri strains to inhibit brewer's yeasts S. cerevisiae inoculated at a very high level (much higher than what it would be as contaminants). Thus, even better inhibition is expected when Saccharomyces spp. is present as a contaminant only.

### Example 2: Inhibition of Saccharomyces pastorianus

### 2.1 Preparation of Samples

Saccharomyces pastorianus (SmartBev, Chr. Hansen A/S, Denmark) was cultivated in malt media (8° P, prepared from Barlex 7215, Harboes Bryggeri A/S, Denmark; pH=4.4) at 25° C. at 150 rpm for 48 hours prior to inoculation.

Wort of 8°P, 13 IBU was prepared from hopped (Magnum 12.9% AA) malt extract (Barlex 7215) and pH adjusted to 4.4. It was divided into 200 mL volume per sample in bluecap bottles. The samples were inoculated with or without various Pichia kluyveri strains simultaneously with S. pastorianus (SmartBev, Chr. Hansen A/S, Denmark). For an overview of the inoculations see Table 3. The fermentations were incubated at 18° C. at 120 rpm for 10 days. Sample of 10 mL was collected on day 1, 3 and 6 after inoculation and subsequently frozen at −50° C.

TABLE 3
Overview of fermentations and inoculation regime
with P. kluyveri and S. pastorianus

| | | 7 | | | |
|---|---|---|---|---|---|
| Microorganism | Strain | Control | 8 | 9 | 10 |
| Pichia kluyveri | DSM 28484 | | 1E+05 | | |
| Pichia kluyveri | DSM 34278 | | | 1E+05 | |
| Pichia kluyveri | PK-KR1 | | | | 1E+05 |
| Saccharomyces | SmartBev | 1E+03 | 1E+03 | 1E+03 | 1E+03 |
| pastorianus | | | | | |

### 2.2 Analysis of Yeast Growth

Yeast growth analysis was carried out as described under 1.2.

### 2.3 Results

In total, 12 samples belonging to the different fermentations and controls taken at days 1, 3 and 6 were analyzed. To quantify S. pastorianus in the experiment conducted on the fermentations, qPCR primers SI4F/SI7R and S-probe as described earlier were used to amplify appropriate product size located on the ITS rRNA gene. The specificity of the primers and probe was validated by qPCR using genomic DNA from all yeast listed in Table 3. The assay displayed excellent efficiency (95% amplification efficiency, R² 0.998) and specificity for S. pastorianus, whereas the various Pichia kluyveri strains remain under the lowest reliable detection limit.

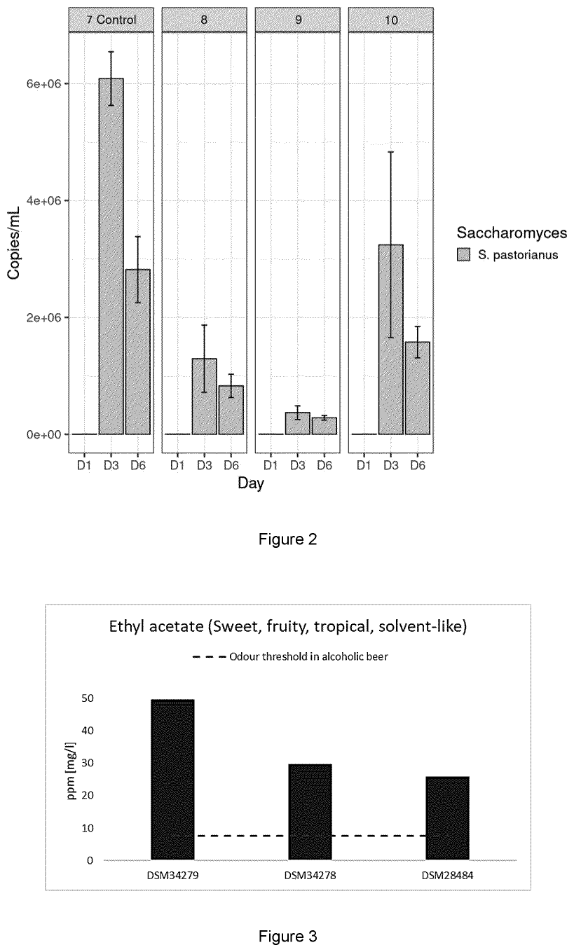

FIG. 2 shows the total concentration of S. pastorianus expressed as gene copies/mL found in the fermentations after one day, three days and 6 days. A decrease of Saccharomyces pastorianus growth between Day 3 and Day 6 was observed for various Pichia kluyveri strains, compared to the control batch (without Pichia kluyveri). These results show the effective use of various Pichia kluyveri strains to inhibit yeast S. pastorianus.

### Example 3: Preparation of non-alcoholic beer using Pichia kluyveri strains DSM 34278, DSM 34279 and DSM28484

### 2.1 Preparation of Non-Alcoholic Beer

6.5 kg of Pilsner malt was added to the mash kettle containing 19.5 L water at 68° C. and kept at this temperature for 30 min. The mash (malt and water) was heated to 72° C. and kept at this temperature for 10 min before being heated to 78° C. and kept at this temperature for 5 minutes. The mash was then filtered and sparged with 40.7 L water, the liquid was transferred to the boil kettle, and heated to boiling. 22 g of hops with 12.8% alpha acid content was added. Boiling lasted for 60 min. The resulting wort was transferred to 5 L plastic jerry cans and stored in a cold room (<5° C.).

5 liter of wort was adjusted to pH 5.3 and eight 500 ml blue cap bottles was filled with 400 ml of the pH adjusted wort. The blue cap bottles with pH adjusted wort were pasteurized by placing them in a water bath at 90° C. for 30 minutes followed by a cooling to room temperature (approx. 20° C.). After cooling they were inoculated with DSM34278, DSM 34279 or DSM 28484 (duplicates) to a cell count of 1E+05 cells/ml. Two of the blue cap bottles were not inoculated and served as negative controls. The eight blue cap bottles were transferred to a shaking incubator set to 18° C. and 100 rpm and kept there for 5 days. The non-alcoholic beer fermented with the yeasts all had an alcohol content ranging from 0.2-0.3% vol.-%.

### 3.2 Analysis of Volatile Compounds

Samples were analyzed by Gas chromatography-mass spectrometry GC-MS at Laboratorio de Análisis del Aroma y Enología (LAAE) at the University of Zaragoza, Spain, for major volatile compounds and free volatile sulphur by GC-MS.

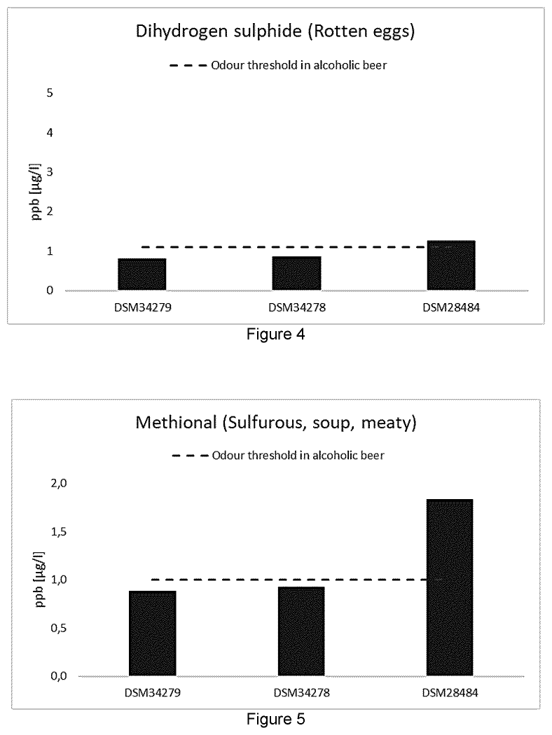

FIGS. 3-5 show the results for ethyl acetate (which imparts sweet fruity and tropical flavor) and off-flavor compounds dihydrogen sulphide (rotten egg smell) and methional (sulturous soup meaty smell). The dotted line indicates odor detection threshold (the lowest concentration of a certain odor compound that is perceivable by the human sense of smell) for the respective compound in alcoholic beer. The values for ethyl acetate and dihydrogen sulphide were retrieved on www.aroxa.com as of Mar. 18, 2022. The values for methional was provided by LAAE.

These results show that the Pichia kluyveri strains DSM 34278 and DSM 34279 produced higher ethyl acetate and less dihydrogen sulphide and methional (below odor threshold) than DSM 28484, indicating better flavor profile.

### Deposit and Expert Solution

The applicant requests that a sample of the deposited microorganisms stated below may only be made available to an expert, subject to available provisions governed by Industrial Property Offices of States Party to the Budapest Treaty, until the date on which the patent is granted.

TABLE 4
The applicant has made the following deposits at a Depositary institution
having acquired the status of international depositary authority
under the Budapest Treaty on the International Recognition of the
Deposit of Microorganisms for the Purposes of Patent Procedure:
Leibniz Institute DSMZ-German Collection of Microorganisms and
Cell Cultures Inhoffenstr. 7B, 38124 Braunschweig, Germany.

| Strain | Accession No. | Deposit date |
|---|---|---|
| Pichia kluyveri DSM 28484 | DSM 28484 | 2014 Mar. 5 |
| Pichia kluyveri DSM 34278 | DSM 34278 | 2022 May 31 |
| Pichia kluyveri DSM 34279 | DSM 34279 | 2022 May 31 |

1. (canceled)

2. A method of inhibiting or delaying growth of Saccharomyces spp. in a beverage with alcohol content of less than 4.2 vol.-%, comprising the step of adding an effective amount of one or more Pichia kluyveri strains, wherein the Pichia kluyveri strain inhibits the growth of S. cerevisiae or S. pastorianus by at least 40% when inoculated simultaneously with the Pichia kluyveri strain, compared to an otherwise identical sample which does not comprise the Pichia kluyveri strain, wherein the Pichia kluyveri strain is not PK-KR1.

3. The method of claim 2, wherein the beverage is a beverage with alcohol content less than 1.2 vol.-%.

4. The method of claim 2, wherein the beverage is a beverage with alcohol content of less than 0.5 vol.-%, and most preferably less than 0.05 vol.-%.

5. (canceled)

6. The method of claim 2, wherein the Pichia kluyveri strain is selected from the group consisting of strains deposited as DSM 34278 and DSM 34279 and a mutant thereof.

7. The method of claim 2, wherein the growth of one or more of Saccharomyces cerevisiae, Saccharomyces carlsbergensis, Saccharomyces eubayanus, Saccharomyces bayanus, Saccharomyces uvarum, Saccharomyce kudriavzevii and/or Saccharomyces pastorianus is inhibited.

8. The method of claim 2, wherein the one or more Pichia kluyveri strains is added to the beverage at a concentration of at least 10³ CFU/ml.

9. The method of claim 2, wherein the beverage is a wine, cider, tea, fruit beverage or beer with alcohol content of less than 4.2 vol.-%.

10. The method of claim 2, wherein the growth of Saccharomyces spp. is inhibited by at least 50%.

11. The method of claim 2, wherein the Pichia kluyveri strain inhibits the growth of Saccharomyces spp. by at least 40% when 10³ CFU/ml Saccharomyces spp. is inoculated simultaneously with 10⁵ CFU/ml Pichia kluyveri strain, compared to an otherwise identical sample which does not comprise the Pichia kluyveri strain.

12. Yeast of the species Pichia kluyveri deposited as DSM 34278, DSM 34279, or a functionally equivalent mutant Pichia kluyveri obtainable from the deposited yeast.

13. (canceled)

14. A composition comprising the Pichia kluyveri strain according to claim 11.

15. A beverage with an alcohol content of less than 4.2 vol.-% comprising the Pichia kluyveri strain according to claim 11.

16. The composition of claim 14, wherein the Pichia kluyveri strain is present in the composition at concentration of at least 10⁶ CFU/g.

17. The composition of claim 14, wherein the composition is in liquid, frozen, or dried form.

18. The composition of claim 14, wherein the composition further comprises one or more cryoprotectants, lyoprotectants, antioxidants, nutrients, fillers, flavorants or mixtures thereof.


SEQUENCE LISTING (3 sequences)

SEQ ID NO: 1 - DNA, 19 bp, synthetic construct
  source 1..19: mol_type = other DNA
attgctggccttttcattg

SEQ ID NO: 2 - DNA, 22 bp, synthetic construct
  source 1..22: mol_type = other DNA
cgcctagacgctctcttcttat

SEQ ID NO: 3 - DNA, 20 bp, synthetic construct
  source 1..20: mol_type = other DNA
gccgcagttggtaaaaccta